(19) United States
(12) Patent Application Publication
(10) Pub. No.: US 2022/0298019 A1
(43) Pub. Date: Sep. 22, 2022

(54) Direct Synthesis of Aluminosilicate Zeolitic Materials of the IWR Framework Structure Type and their Use in Catalysis

(71) Applicant: BASF SE, Ludwigshafen am Rhein (DE)

(72) Inventors: Andrei-Nicolae Parvulescu, Silver Lake, OH (US); Feng-Shou Xiao, Hangzhou (CN); Xiangju Meng, Hangzhou (CN); Qinming Wu, Hangzhou (CN); Ulrich Mueller, Ludwigshafen (DE); Toshiyuki Yokoi, Tokyo (JP); Weiping Zhang, Dalian (CN); Ute Kolb, Mainz (DE); Bernd Marler, Bochum (DE); Dirk De Vos, Leuven (DE); Xin Hong, Hangzhou (CN)

(21) Appl. No.: 17/596,209

(22) PCT Filed: Jun. 5, 2020
(86) PCT No.: PCT/CN2020/094663

§ 371 Date: Dec. 6, 2021

(30) Foreign Application Priority Data
Jun. 6, 2019 (CN) PCT/CN2019/090361

(51) Int. Cl. C01B 39/46; C07C 1/20

(52) U.S. Cl. CPC C01B 39/46; C07C 1/20; C07C 2529/70

(57) ABSTRACT

The present invention relates to a zeolitic material having the IWR type framework structure, wherein the zeolitic material comprises YO₂ and X₂O₃ in its framework structure, wherein Y is a tetravalent element and X is a trivalent element, and wherein the framework structure of the zeolitic material comprises less than 5 weight-% weight-% of Ge calculated as GeO₂ and based on 100 weight-% weight-% of YO₂ contained in the framework structure, and less than 5 weight-% weight-% of B calculated as B₂O₃ and based on 100 weight-% weight-% of X₂O₃ contained in the framework structure. Further, the present invention relates to a process for preparing a zeo-litic material having the IWR type framework structure, wherein the zeolitic material comprises YO₂ and X₂O₃ in its framework structure, wherein Y is a tetravalent element and X is a trivalent element.

### TECHNICAL FIELD

The present invention relates to a process for the preparation of a zeolitic material as well as to a zeolitic material having the IWR-type framework structure as such and as obtainable from the inventive process. Furthermore, the present invention relates to the use of the inventive zeolitic materials in specific applications.

### INTRODUCTION

The ITQ-24 zeolite with IWR structure was first synthesized in the presence of germanium species using hexamethonium as an organic template. Thus, EP 1 609 758 B1 discloses the zeolite Ge-ITQ-24 which is obtained with Ge as a tetravalent element in addition to Si in its zeolitic framework. Although said document claims that the tetravalent element of the framework structure may be selected from a list of elements including Si, said document contains no teaching which would have allowed the skilled person to obtain compounds with a framework structure devoid of Ge.

Because of its unique three dimensional 12×10×10-membered ring pore structure (aperture size of 5.8×6.8, 4.6×5.3, and 4.6×5.3 Å), ITQ-24 has attracted much attention. However, when a large amount of germanium species exist in the IWR framework, its thermal and hydrothermal stability is remarkably reduced. In addition, the use of germanium species in the synthesis is costly, which strongly hinders the applications of IWR zeolite as heterogeneous catalysts. To solve this problem, Cantin, A. et al. in J. Am. Chem. Soc. 2006, 128, pp. 4216-4217 describe a Ge-free route for the synthesis of IWR zeolite by introduction of boron species instead of germanium due to very close Si—O—Ge angles to those of Si—O—B, wherein with the assistance of seeds, pure silica IWR could also be synthesized. However, from the view of industrial applications, the aluminosilicate IWR zeolite would be more attractive due to its strong acidity and superior thermal and hydrothermal stabilities. Due to the lack of a direct synthesis of aluminosilicate IWR zeolite, Shamzhy, M. et al. in Catal. Today 2015, 243, 76-84 describe a post-synthesis treatment for alumination of borogermanosilicate IWR zeolite.

Further, there have been many successful examples for synthesis of aluminosilicate zeolites (ITQ-22, TNU-9, IM-5, SSZ-74, EMM-23) employing pyrrolidine-based cations as efficient organic templates. CN 106698456 A, on the other hand, relates to the synthesis of the zeolite Al-ITQ-13 having the ITH type framework structure, wherein a linear polyquarternary ammonium organic template is employed as the structure directing agent. Simancas R. et al. in Science 2010, 330, pp. 1219-1222, for its part, concerns the synthesis of the zeolite ITQ-47 using phosphazenes as the structure directing agent.

Thus, there remains the need for a direct synthesis of an aluminosicate having the IWR frame-work structure, in particular for obtaining a material which is free of germanium. Furthermore, despite the large variety of existing zeolite structures and specific zeolitic materials, an ongoing need remains for the synthesis of new zeolitic materials with unique physical and chemical characteristics, in particular in view of their increased use in catalytic applications.

### DETAILED DESCRIPTION

It was therefore the object of the present invention to provide a new zeolitic material and a method for its synthesis. Furthermore, it was the object of the present invention to provide a new zeolitic material for catalytic applications, in particular for heterogeneous catalysis, and particularly for the conversion of oxygenates to olefins. Thus, it has surprisingly been found that a zeolitic material of the IWR framework-type structure may be directly synthesized using the p-xylylene-bis((N-methyl)N-pyrrolidinium) organotemplate as the structure directing agent. In particular, it has quite unexpectedly been found that using the aforementioned organotemplate, a zeolitic material of the IWR framework type containing Si as the tetravalent element of the zeolitic framework in addition to Al as the trivalent element may be directly obtained. Furthermore, it has surprisingly been found that the zeolitic materials of the present invention display unique properties in catalysis, and in particular in the conversion of oxygenates to olefins, wherein in the conversion of methanol to olefins excellent C3 selectivities may be achieved. Furthermore, the inventive zeolitic materials display much higher thermal and in particular hydrothermal stabilities than conventional Ge—Al-IWR zeolites.

Therefore, the present invention relates to a zeolitic material having the IWR type framework structure, preferably obtainable and/or obtained according to the process of any one of the embodiments disclosed herein, wherein the zeolitic material comprises YO₂ and X₂O₃ in its framework structure, wherein Y is a tetravalent element and X is a trivalent element, and wherein the framework structure of the zeolitic material comprises less than 5 weight-% of Ge calculated as GeO₂ and based on 100 weight-% of YO₂ contained in the framework structure, and less than 5 weight-% of B calculated as B₂O₃ and based on 100 weight-% of X₂O₃ contained in the framework structure.

Further, the present invention relates to a process for the preparation of a zeolitic material having the IWR type framework structure, preferably of a zeolitic material according to any one of the embodiments disclosed herein, wherein the process comprises
(1) preparing a mixture comprising one or more organotemplates as structure directing agents, one or more sources of YO₂, one or more sources of X₂O₃, and a solvent system;
(2) heating the mixture obtained in (1) for crystallizing a zeolitic material having the IWR type framework structure comprising YO₂ and X₂O₃ in its framework structure;
wherein the one or more organotemplates comprise an organodication of the formula (I):

R³R⁵R⁶N⁺—R¹-Q-R²—N⁺R⁴R⁷R⁸ (I);
wherein R¹ and R² independently from one another stand for (C₁-C₃)alkylene, preferably for C₁ or C₂ alkylene, more preferably for methylene or ethylene, and more preferably for methylene;
   wherein Q stands for C₆-arylene, preferably for 1,4-C₆-arylene, and more preferably for benzene-1,4-diyl;
wherein R³ and R⁴ independently from one another stand for (C₁-C₄)alkyl, preferably (C₁-C₃)alkyl, more preferably for methyl or ethyl, and more preferably for methyl;
wherein R⁵, R⁶, R⁷, and R⁸ independently from one another stand for (C₁-C₆)alkyl, preferably (C₁-C₅)alkyl, more preferably (C₁-C₄)alkyl, more preferably (C₁-C₃)alkyl, more preferably for ethyl, isopropyl, or n-propyl, and more preferably for ethyl or n-propyl.

Yet further, the present invention relates to a zeolitic material obtainable and/or obtained from the process of any one of the embodiments disclosed herein.

Yet further, the present invention relates to a method for the conversion of oxygenates to olefins comprising
(i) providing a catalyst according to any one of the embodiments disclosed herein;
(ii) providing a gas stream comprising one or more oxygenates and optionally one or more olefins and/or optionally one or more hydrocarbons;
(iii) contacting the catalyst provided in (i) with the gas stream provided in (ii) and converting one or more oxygenates to one or more olefins and optionally to one or more hydrocarbons;
(iv) optionally recycling one or more of the one or more olefins and/or of the one or more hydrocarbons contained in the gas stream obtained in (iii) to (ii).

Yet further, the present invention relates to a use of a zeolitic material according to any one of the embodiments disclosed herein as a molecular sieve, as an adsorbent, for ion-exchange, or as a catalyst and/or as a catalyst support, preferably as a catalyst for the selective catalytic reduction (SCR) of nitrogen oxides NOₓ; for the oxidation of NH₃, in particular for the oxidation of NH₃ slip in diesel systems; for the decomposition of N₂O; as an additive in fluid catalytic cracking (FCC) processes; and/or as a catalyst in organic conversion reactions, preferably as a hydrocracking catalyst, as an alkylation catalyst, as an isomerization catalyst, or as a catalyst in the conversion of alcohols to olefins, and more preferably in the conversion of oxygenates to olefins.

It is preferred that the zeolitic material comprises less than 3 weight-% of Ge calculated as GeO₂ and based on 100 weight-% of YO₂ contained in the framework structure, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%. Thus, it is particularly preferred that the zeolitic material, preferably the framework structure of the zeolitic material, is substantially free of Ge.

It is preferred that the zeolitic material comprises less than 3 weight-% of B calculated as B₂O₃ and based on 100 weight-% of X₂O₃ contained in the framework structure, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%. Thus, it is particularly preferred that the zeolitic material, preferably the framework structure of the zeolitic material, is substantially free of B.

It is preferred that Y is selected from the group consisting of Si, Sn, Ti, Zr, and mixtures of two or more thereof, Y more preferably being Si and/or Ti, wherein Y is more preferably Si.

It is preferred that X is selected from the group consisting of Al, In, Ga, Fe, and mixtures of two or more thereof, X more preferably being Al and/or Ga, wherein X is more preferably Al.

It is preferred that the YO₂:X₂O₃ molar ratio of the framework structure of the zeolitic material is in the range of from 5 to 1,000, more preferably of from 10 to 700, more preferably of from 30 to 500, more preferably of from 50 to 400, more preferably of from 100 to 350, more preferably of from 150 to 310, more preferably of from 200 to 290, and more preferably of from 250 to 270.

In accordance with the above, it is particularly preferred that Y is Si and X is Al. Thus, it is preferred that 95 or more weight-% of the zeolitic material consists of Si, Al, O, and H, calculated based on the total weight of the zeolitic material, preferably 95 to 100 weight-%, more preferably 97 to 100 weight-%, more preferably 99 to 100 weight-%.

The zeolitic material may comprise one or more further components. In particular, the zeolitic material may comprise one or more further components at the ion-exchange sites of the framework structure of the zeolitic material. In other words, the zeolitic material may be ion-exchanged. It is preferred that the zeolitic material comprises one or more metal cations M at the ion-exchange sites of the framework structure of the zeolitic material, wherein the one or more metal cations M are preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably from the group consisting of Sr, Cr, Mg, Ca, Mo, Fe, Co, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Cr, Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, and more preferably from the group consisting of Fe, Cu, Mg, Ca, Zn, Mo, La, Ce, Pr, Nd, Sm, Eu, Gd, Tb, Dy, Ho, Er, Tm, Yb, Lu, Y, Sc, and mixtures of two or more thereof.

In the case where the the zeolitic material comprises one or more metal cations M at the ion-exchange sites of the framework structure of the zeolitic material, it is preferred that the zeolitic material comprises the one or more metal cations M in an amount in the range of from 0.01 to 10 weight-% based on 100 weight-% of Si in the zeolitic material calculated as SiO₂, more preferably in the range of from 0.05 to 7 weight-%, more preferably in the range of from 0.1 to 5 weight-%, more preferably in the range of from 0.5 to 4.5 weight-%, more preferably in the range of from 1 to 4 weight-%, more preferably in the range of from 1.5 to 3.5 weight-%, and more preferably in the range of from 2 to 3 weight-%.

In accordance with the above, it is particularly preferred that Y is Si and X is Al. Thus, it is preferred that 95 or more weight-% of the zeolitic material consists of Si, Al, O, H, and the one or more metal cations M, calculated based on the total weight of the zeolitic material, preferably 95 to 100 weight-%, more preferably 97 to 100 weight-%, more preferably 99 to 100 weight-%.

As disclosed above, it is preferred that Y is Si. In the case where Y is Si, it is preferred that the ²⁹Si MAS NMR of the zeolitic material comprises:
a first peak in the range of from −99 to −102 ppm, preferably of from −99.5 to −101.5 ppm, more preferably of from −100 to −101.2 ppm, more preferably of from −100.3 to −100.9 ppm, and even more preferably of from −100.5 to −100.7 ppm;
a second peak in the range of from −105.5 to −108 ppm, preferably of from −106 to −107.5 ppm, more preferably of from −106.3 to −107.3 ppm, more preferably of from −106.5 to −107.1 ppm, and even more preferably of from −106.7 to −106.9 ppm; and
a third peak in the range of from −112.5 to −115 ppm, preferably of from −113 to −114.5 ppm, more preferably of from −113.3 to −114.3 ppm, more preferably of from −113.5 to −114.1 ppm, and even more preferably of from −113.7 to −113.9 ppm;
wherein preferably the ²⁹Si MAS NMR of the zeolitic material comprises only three peaks in the range of from −80 to −130 ppm.

As disclosed above, it is preferred that X is Al. In the case where X is Al, it is preferred that the ²⁷Al MAS NMR of the zeolitic material comprises:
a peak in the range of from 55 to 58 ppm, preferably of from 55.5 to 57.5 ppm, more preferably of from 56 to 57 ppm, more preferably of from 56.6 to 56.8 ppm, and even more preferably of from 56.4 to 56.6 ppm,
wherein preferably the ²⁷Al MAS NMR of the zeolitic material comprises a single peak in the range of from −40 to −140 ppm.

It is preferred that the BET surface area of the zeolitic material determined according to ISO 9277:2010 ranges from 100 to 850 m²/g, more preferably from 300 to 800 m²/g, more preferably from 400 to 750 m²/g, more preferably from 500 to 700 m²/g, more preferably from 530 to 650 m²/g, more preferably from 550 to 620 m²/g, more preferably from 570 to 590 m²/g.

It is preferred that the micropore volume of the zeolitic material determined according to ISO 15901-1:2016 is in the range of from 0.1 to 0.5 cm³/g, more preferably from 0.15 to 0.4 cm3/g, more preferably from 0.2 to 0.35 cm³/g, more preferably from 0.23 to 0.32 cm³/g, more preferably from 0.25 to 0.3 cm³/g, and more preferably from 0.26 to 0.28 cm³/g.

It is preferred that the zeolitic material is ITQ-24.

Further, the present invention relates to a process for the preparation of a zeolitic material having the IWR type framework structure, preferably of a zeolitic material according to any of the embodiments disclosed herein, wherein the process comprises
(1) preparing a mixture comprising one or more organotemplates as structure directing agents, one or more sources of YO₂, one or more sources of X₂O₃, and a solvent system;
(2) heating the mixture obtained in (1) for crystallizing a zeolitic material having the IWR type framework structure comprising YO₂ and X₂O₃ in its framework structure;
wherein the one or more organotemplates comprise an organodication of the formula (I):

R³R⁵R⁶N⁺—R¹-Q-R²—N⁺R⁴R⁷R⁸ (I);
wherein R¹ and R² independently from one another stand for (C₁-C₃)alkylene, preferably for C₁ or C₂ alkylene, more preferably for methylene or ethylene, and more preferably for methylene;
wherein Q stands for C₆-arylene, preferably for 1,4-C₆-arylene, and more preferably for benzene-1,4-diyl;
wherein R³ and R⁴ independently from one another stand for (C₁-C₄)alkyl, preferably (C₁-C₃)alkyl, more preferably for methyl or ethyl, and more preferably for methyl;
wherein R⁵, R⁶, R⁷, and R⁸ independently from one another stand for (C₁-C₆)alkyl, preferably (C₁-C₅)alkyl, more preferably (C₁-C₄)alkyl, more preferably (C₁-C₃)alkyl, more preferably for ethyl, isopropyl, or n-propyl, and more preferably for ethyl or n-propyl.

It is preferred that the alkyl groups R⁵ and R⁶ are bound to one another to form one common alkylene chain, more preferably a (C₅-C₇)alkylene chain, more preferably a (C₅-C₆)alkylene chain, more preferably a pentylene or hexylene chain, and more preferably a pentylene chain.

It is preferred that the alkyl groups R⁷ and R⁸ are bound to one another to form one common alkylene chain, more preferably a (C₅-C₇)alkylene chain, more preferably a (C₅-C₆)alkylene chain, more preferably a pentylene or hexylene chain, and more preferably a pentylene chain.

It is preferred that the organodication of the formula (I) has the formula (II):

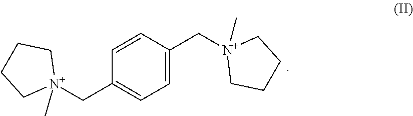 [SMILES: C[N+]1(Cc2ccc(C[N+]3(C)CCCC3)cc2)CCCC1]

It is preferred that the one or more organotemplates are provided as salts, preferably as one or more salts selected from the group consisting of halides, sulfate, nitrate, phosphate, acetate, hydroxide, and mixtures of two or more thereof, more preferably from the group consisting of bromide, chloride, hydroxide, sulfate, and mixtures of two or more thereof, wherein more preferably the one or more organotemplates are provided as hydroxides and/or bromides, and more preferably as hydroxides.

It is preferred that Y is selected from the group consisting of Si, Sn, Ti, Zr, Ge, and mixtures of two or more thereof, Y more preferably being Si and/or Ti, wherein Y is more preferably Si.

It is preferred that X is selected from the group consisting of Al, B, In, Ga, and mixtures of two or more thereof, more preferably from the group consisting of Al, B, Ga, and mixtures of two or more thereof, X more preferably being Al and/or B, wherein X is more preferably Al.

It is preferred that the mixture prepared in (1) further comprises seed crystals, wherein the seed crystals more preferably comprise one or more all-silica zeolitic materials having the IWR type framework structure, wherein more preferably the seed crystals comprise all-silica ITQ-24, wherein more preferably one or more all-silica zeolitic materials having the IWR type framework structure is employed as the seed crystals, wherein more preferably all-silica ITQ-24 is employed as the seed crystals.

It is preferred that the mixture prepared in (1) further comprises seed crystals, wherein the seed crystals preferably comprise one or more zeolitic materials having the IWR type framework structure, and more preferably one or more zeolitic materials according to any one of the embodiments disclosed herein, wherein more preferably one or more zeolitic materials having the IWR type framework structure is employed as the seed crystals, wherein more preferably one or more zeolitic materials according to any one of the embodiments disclosed herein is employed as the seed crystals.

In the case where the mixture prepared in (1) further comprises seed crystals, it is preferred that the amount of seed crystals comprised in the mixture prepared in (1) is in the range of from 0.1 to 15 mol % based on 100 mol % of the one or more sources of YO₂ calculated as YO₂, more preferably from 0.5 to 12 mol %, more preferably from 1 to 10 mol %, more preferably from 2 to 8 mol %, more preferably from 3 to 7 mol %, and more preferably from 5 to 6 mol %.

It is preferred that the mixture prepared in (1) and heated in (2) contains less than 5 weight-% of Ge calculated as GeO₂ and based on 100 weight-% of the one or more sources of YO₂ calculated as YO₂, more preferably less than 3 weight-%, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%. Thus, it is preferred that the mixture prepared in (1) and heated in (2) is substantially free of Ge.

It is preferred that the mixture prepared in (1) and heated in (2) contains less than 5 weight-% of B calculated as B₂O₃ and based on 100 weight-% of the one or more sources of X₂O₃ calculated as X₂O₃, more preferably less than 3 weight-%, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%. Thus, it is preferred that the mixture prepared in (1) and heated in (2) is substantially free of B.

It is preferred that the X₂O₃:YO₂ molar ratio of the one or more sources of X₂O₃ calculated as X₂O₃ to the one or more sources of YO₂ calculated as YO₂ in the mixture prepared in (1) and heated in (2) is in the range of from 5 to 1,500, more preferably of from 10 to 1,200, more preferably of from 30 to 1,000, more preferably of from 50 to 900, more preferably of from 100 to 800, more preferably of from 200 to 700, more preferably of from 250 to 600, more preferably of from 300 to 500, and more preferably of from 350 to 400.

It is preferred that the organotemplate:YO₂ molar ratio of the one or more organotemplates to the one or more sources of YO₂ calculated as YO₂ in the mixture prepared in (1) and heated in (2) is in the range of from 0.01 to 1.5, more preferably from 0.05 to 1.2, more preferably from 0.1 to 0.9, more preferably from 0.15 to 0.7, more preferably from 0.2 to 0.5, and more preferably from 0.25 to 0.3.

The mixture prepared in (1) may comprise further components. It is preferred that the mixture prepared in (1) further comprises one or more sources of fluoride. In the case where the mixture prepared in (1) further comprises one or more sources of fluoride, it is preferred that the fluoride:YO₂ molar ratio of the one or more sources of fluoride calculated as the element to the one or more sources of YO₂ calculated as YO₂ in the mixture prepared in (1) and heated in (2) is in the range of from 0.01 to 2, more preferably from 0.05 to 1.5, more preferably from 0.1 to 1, more preferably from 0.3 to 0.8, and more preferably from 0.5 to 0.6.

In the case where the mixture prepared in (1) further comprises one or more sources of fluoride, it is preferred that the one or more sources of fluoride is selected from fluoride salts, HF, and mixtures of two or more thereof, more preferably from the group consisting of alkali metal fluoride salts, HF, and mixtures of two or more thereof, wherein more preferably the one or more sources of fluoride comprise HF, wherein more preferably HF is employed as the one or more sources of fluoride.

It is preferred that heating in (2) is conducted for a duration in the range of from 10 min to 10 d, more preferably from 30 min to 9 d, more preferably from 1 h to 8 d, more preferably from 2 h to 7 d, and more preferably from 3 h to 6 d, more preferably from 6 h to 5.5 d, more preferably from 0.5 to 5 d, more preferably from 1 d to 4.5 d, more preferably from 2 d to 4 d, and more preferably from 2.5 to 3.5 d.

It is preferred that heating in (2) is conducted at a temperature in the range of from 80 to 220° C., more preferably of from 110 to 200° C., more preferably of from 130 to 190° C., more preferably of from 140 to 180° C., more preferably of from 150 to 170° C., and more preferably of from 155 to 165° C.

It is preferred that heating in (2) is conducted under autogenous pressure, more preferably under solvothermal conditions, more preferably under hydrothermal conditions, wherein preferably heating in (2) is performed in a pressure tight vessel, preferably in an autoclave.

The process for the preparation of a zeolitic material having the IWR type framework structure as disclosed herein may comprise further process steps. It is preferred that the process further comprises
(3) isolating the zeolitic material obtained in (2),
and/or
(4) washing the zeolitic material obtained in (2) or (3),
and/or
(5) calcining the zeolitic material obtained in (2), (3), or (4),
and/or
(6) subjecting the zeolitic material obtained in (2), (3), (4), or (5) to an ion-exchange procedure with one or more metal cations M,
wherein the steps (3) and/or (4) and/or (5) and/or (6) can be conducted in any order, and
wherein one or more of said steps is preferably repeated one or more times.

It is preferred that the process further comprises (6). In the case where the process further comprises (6), it is preferred that the one or more metal cations M are selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably from the group consisting of Sr, Cr, Mg, Ca, Mo, Fe, Co, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Cr, Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, and more preferably from the group consisting of Fe, Cu, Mg, Ca, Zn, Mo, La, Ce, Pr, Nd, Sm, Eu, Gd, Tb, Dy, Ho, Er, Tm, Yb, Lu, Y, Sc, and mixtures of two or more thereof, wherein the one or more metal cations M are located at the ion-exchange sites of the framework structure of the zeolitic material.

It is preferred that the process further comprises (5). In the case where the process further comprises (5), it is preferred that calcination in (5) is conducted for a duration in the range of from 0.5 to 15 h, more preferably of from 1 to 10 h, more preferably of from 2 to 8 h, more preferably of from 3 to 7 h, more preferably of from 3.5 to 6.5 h, more preferably of from 4 to 6 h, and more preferably of from 4.5 to 5.5 h.

Further in the case where the process comprises (5), it is preferred that calcination in (5) is conducted at a temperature in the range of from 300 to 800° C., more preferably of from 350 to 700° C., more preferably of from 400 to 650° C., more preferably of from 450 to 600° C., and more preferably of from 500 to 550° C.

It is preferred that the one or more sources for YO₂ comprises one or more compounds selected from the group consisting of fumed silica, silica hydrosols, reactive amorphous solid silicas, silica gel, silicic acid, water glass, sodium metasilicate hydrate, sesquisilicate, disilicate, colloidal silica, silicic acid esters, and mixtures of two or more thereof, more preferably from the group consisting of silica hydrosols, silica gel, silicic acid, water glass, sodium metasilicate hydrate, sesquisilicate, disilicate, colloidal silica, tetra(C₁-C₄)alkylorthosilicate, and mixtures of two or more thereof, more preferably from the group consisting of silica hydrosols, silicic acid, tetra(C₂-C₃)alkylorthosilicate, and mixtures of two or more thereof, wherein more preferably the one or more sources for YO₂ comprises tetraethylorthosilicate, wherein more preferably tetraethylorthosilicate is used as the one or more sources for YO₂.

It is preferred that the one or more sources for X₂O₃ comprises one or more compounds selected from the group consisting of alumina, aluminates, aluminum salts, and mixtures of two or more thereof, more preferably from the group consisting of alumina, aluminum salts, and mixtures of two or more thereof, more preferably from the group consisting of alumina, aluminum tri(C₁-O₅)alkoxide, AlO(OH), Al(OH)₃, aluminum halides, preferably aluminum fluoride and/or chloride and/or bromide, more preferably aluminum fluoride and/or chloride, and even more preferably aluminum chloride, aluminum sulfate, aluminum phosphate, aluminum fluorosilicate, and mixtures of two or more thereof, more preferably from the group consisting of aluminum tri(C₂-C₄)alkoxide, AlO(OH), Al(OH)₃, aluminum chloride, aluminum sulfate, aluminum phosphate, and mixtures of two or more thereof, more preferably from the group consisting of aluminum tri(C₂-C₃)alkoxide, AlO(OH), Al(OH)₃, aluminum chloride, aluminum sulfate, and mixtures of two or more thereof, more preferably from the group consisting of aluminum tripropoxides, AlO(OH), aluminum sulfate, and mixtures of two or more thereof, wherein more preferably the one or more sources for X₂O₃ comprises aluminum triisopropoxide, and wherein more preferably aluminum triisopropoxide is used as the one or more sources for X₂O₃.

It is preferred that the solvent system is selected from the group consisting of optionally branched (C₁-C₄)alcohols, distilled water, and mixtures thereof, more preferably from the group consisting of optionally branched (C₁-C₃)alcohols, distilled water, and mixtures thereof, more preferably from the group consisting of methanol, ethanol, distilled water, and mixtures thereof, wherein more preferably the solvent system comprises distilled water, wherein more preferably the solvent system consists of distilled water.

In the case where the solvent system comprises, or consists of, distilled water, it is preferred that the H₂O:YO₂ molar ratio of H₂O to the one or more sources of YO₂ calculated as YO₂ in the mixture prepared in (1) and heated in (2) is in the range of from 0.5 to 15, more preferably from 1 to 10, more preferably from 1.5 to 5, and more preferably from 2 to 3.

Further, the present invention relates to a zeolitic material obtainable and/or obtained from the process of any one of the embodiments disclosed herein.

Further, the present invention relates to a method for the conversion of oxygenates to olefins comprising
(i) providing a catalyst according to any one of the embodiments disclosed herein;
(ii) providing a gas stream comprising one or more oxygenates and optionally one or more olefins and/or optionally one or more hydrocarbons;
(iii) contacting the catalyst provided in (i) with the gas stream provided in (ii) and converting one or more oxygenates to one or more olefins and optionally to one or more hydrocarbons;
(iv) optionally recycling one or more of the one or more olefins and/or of the one or more hydrocarbons contained in the gas stream obtained in (iii) to (ii).

It is preferred that the catalyst is provided as a fixed bed or as a fluidized bed.

It is preferred that the gas stream provided in (ii) comprises one or more oxygenates selected from the group consisting of aliphatic alcohols, ethers, carbonyl compounds and mixtures of two or more thereof, more preferably from the group consisting of (C₁-C₆) alcohols, di(C₁-C₃)alkyl ethers, (C₁-C₆) aldehydes, (C₂-C₆) ketones and mixtures of two or more thereof, more preferably consisting of (C₁-C₄) alcohols, di(C₁-C₂)alkyl ethers, (C₁-C₄) aldehydes, (C₂-C₄) ketones and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone and mixtures of two or more thereof, more preferably from the group consisting of methanol, ethanol, dimethyl ether, diethyl ether, ethyl methyl ether and mixtures of two or more thereof, the gas stream more preferably comprising methanol and/or dimethyl ether, and more preferably dimethyl ether or a mixture of dimethyl ether and methanol.

It is preferred that the content of oxygenates in the gas stream provided in (ii) is in the range from 2 to 100% by volume based on the total volume, more preferably from 3 to 99% by volume, more preferably from 4 to 95% by volume, more preferably from 5 to 80% by volume, more preferably from 6 to 50% by volume, more preferably from 7 to 40% by volume, more preferably from 8 to 30% by volume, more preferably from 9 to 20% by volume, and more preferably from 10 to 15% by volume.

The gas stream provided in (ii) may further comprise water. It is preferred that the water content in the gas stream provided in (ii) is in the range from 5 to 60% by volume, more preferably from 10 to 50% by volume, more preferably from 20 to 45% by volume, and more preferably from 30 to 40% by volume.

It is preferred that the gas stream provided in (ii) further comprises one or more diluting gases. In the case where the gas stream provided in (ii) further comprises one or more diluting gases, it is preferred that the gas stream comprises the one or more diluting gases in an amount in the range of from 0.1 to 90% by volume, more preferably from 1 to 85% by volume, more preferably from 5 to 80% by volume, more preferably from 10 to 75% by volume, more preferably from 20 to 70% by volume, more preferably from 40 to 65% by volume, more preferably from 50 to 60% by volume.

Further in the case where the gas stream provided in (ii) further comprises one or more diluting gases, it is preferred that the one or more diluting gases are selected from the group consisting of H₂O, helium, neon, argon, krypton, nitrogen, carbon monoxide, carbon dioxide, and mixtures of two or more thereof, more preferably from the group consisting of H₂O, argon, nitrogen, carbon dioxide, and mixtures of two or more thereof, wherein more preferably the one or more diluting gases comprise H₂O, wherein more preferably the one or more diluting gases is H₂O.

It is preferred that the contacting according to (iii) is effected at a temperature in the range from 200 to 700° C., more preferably from 250 to 650° C., more preferably from 300 to 600° C., more preferably from 350 to 550° C., more preferably from 400 to 500° C., and more preferably from 425 to 475° C.

It is preferred that the contacting according to (iii) is effected at a pressure in the range from 0.1 to 50 bar, more preferably from 0.3 to 30 bar, more preferably from 0.5 to 20 bar, more preferably from 1 to 15 bar, more preferably from 1.3 to 10 bar, more preferably from 1.5 to 7 bar, more preferably from 1.8 to 5 bar, more preferably from 2.0 to 3.0 bar, more preferably from 2.2 to 2.8 bar, more preferably from 2.4 to 2.6 bar.

It is preferred that the method is a continuous method. In the case where the method is a continuous method, it is preferred that the gas hourly space velocity (GHSV) in the contacting in (iii) is in the range from 500 to 30,000 h⁻¹, more preferably from 1,000 to 20,000 h⁻¹, more preferably from 1,500 to 10,000 h⁻¹, more preferably from 2,000 to 5,000 h⁻¹, more preferably from 2,200 to 3,000 h⁻¹ and more preferably from 2,400 to 2,600 h⁻¹.

It is preferred that the gas stream provided in (ii) comprises the one or more olefins and/or one or more hydrocarbons. In the case where the gas stream provided in (ii) comprises the one or more olefins and/or one or more hydrocarbons, it is preferred that the one or more olefins and/or one or more hydrocarbons comprise one or more selected from the group consisting of ethylene, (C₄-C₇)olefins, (C₄-C₇)hydrocarbons, and mixtures of two or more thereof, and preferably from the group consisting of ethylene, (C₄-C₅)olefins, (C₄-C₅)hydrocarbons, and mixtures of two or more thereof.

It is preferred that one or more olefins and/or one or more hydrocarbons are provided in the gas stream in (ii). It is preferred that one or more olefins and/or one or more hydrocarbons are recycled in the gas stream in (ii). In the case where one or more olefins and/or one or more hydrocarbons are recycled in the gas stream in (ii), it is preferred that the one or more olefins and/or one or more hydrocarbons recycled to (ii) comprise one or more selected from the group consisting of ethylene, (C₄-C₇)olefins, (C₄-C₇)hydrocarbons, and mixtures of two or more thereof, and preferably from the group consisting of ethylene, (C₄-C₅)olefins, (C₄-C₅)hydrocarbons, and mixtures of two or more thereof.

Further, the present invention relates to a use of a zeolitic material according to any one of the embodiments disclosed herein as a molecular sieve, as an adsorbent, for ion-exchange, or as a catalyst and/or as a catalyst support, preferably as a catalyst for the selective catalytic reduction (SCR) of nitrogen oxides NOₓ; for the oxidation of NH₃, in particular for the oxidation of NH₃ slip in diesel systems; for the decomposition of N₂O; as an additive in fluid catalytic cracking (FCC) processes; and/or as a catalyst in organic conversion reactions, preferably as a hydrocracking catalyst, as an alkylation catalyst, as an isomerization catalyst, or as a catalyst in the conversion of alcohols to olefins, and more preferably in the conversion of oxygenates to olefins.

It is preferred that the zeolitic material is used in a methanol-to-olefin process (MTO process), in a dimethylether to olefin process (DTO process), methanol-to-gasoline process (MTG process), in a methanol-to-hydrocarbon process, in a methanol to aromatics process, in a biomass to olefins and/or biomass to aromatics process, in a methane to benzene process, for alkylation of aromatics, or in a fluid catalytic cracking process (FCC process), preferably in a methanol-to-olefin process (MTO process) and/or in a dimethylether to olefin process (DTO process), and more preferably in a methanol-to-propylene process (MTP process), in a methanol-to-propylene/butylene process (MT3/4 process), in a dimethylether-to-propylene process (DTP process), in a dimethylether-to-propylene/butylene process (DT3/4 process), and/or in a dimethylether-to-ethylene/propylene (DT2/3 process).

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Further, it is explicitly noted that the following set of embodiments is not the set of claims determining the extent of protection, but represents a suitably structured part of the description directed to general and preferred aspects of the present invention.
1. A zeolitic material having the IWR type framework structure, preferably obtainable and/or obtained according to the process of any one of embodiments 16 to 42, wherein the zeolitic material comprises YO₂ and X₂O₃ in its framework structure, wherein Y is a tetravalent element and X is a trivalent element, and wherein the framework structure of the zeolitic material comprises less than 5 weight-% of Ge calculated as GeO₂ and based on 100 weight-% of YO₂ contained in the framework structure, and less than 5 weight-% of B calculated as B₂O₃ and based on 100 weight-% of X₂O₃ contained in the framework structure.
2. The zeolitic material of embodiment 1, wherein the zeolitic material comprises less than 3 weight-% of Ge calculated as GeO₂ and based on 100 weight-% of YO₂ contained in the framework structure, preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.
3. The zeolitic material of embodiment 1 or 2, wherein the zeolitic material comprises less than 3 weight-% of B calculated as B₂O₃ and based on 100 weight-% of X₂O₃ contained in the framework structure, preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.
4. The zeolitic material of any one of embodiments 1 to 3, wherein Y is selected from the group consisting of Si, Sn, Ti, Zr, and mixtures of two or more thereof, Y preferably being Si and/or Ti, wherein Y is more preferably Si.
5. The zeolitic material of any one of embodiments 1 to 4, wherein X is selected from the group consisting of Al, In, Ga, Fe, and mixtures of two or more thereof, X preferably being Al and/or Ga, wherein X is more preferably Al.
6. The zeolitic material of any one of embodiments 1 to 5, wherein the YO₂:X₂O₃ molar ratio of the framework structure of the zeolitic material is in the range of from 5 to 1,000, preferably of from 10 to 700, more preferably of from 30 to 500, more preferably of from 50 to 400, more preferably of from 100 to 350, more preferably of from 150 to 310, more preferably of from 200 to 290, and more preferably of from 250 to 270.
7. The zeolitic material of any one of embodiments 1 to 6, wherein the zeolitic material comprises one or more metal cations M at the ion-exchange sites of the framework structure of the zeolitic material, wherein the one or more metal cations M are preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably from the group consisting of Sr, Cr, Mg, Ca, Mo, Fe, Co, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Cr, Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, and more preferably from the group consisting of Fe, Cu, Mg, Ca, Zn, Mo, La, Ce, Pr, Nd, Sm, Eu, Gd, Tb, Dy, Ho, Er, Tm, Yb, Lu, Y, Sc, and mixtures of two or more thereof.
8. The zeolitic material of embodiment 7, wherein the zeolitic material comprises the one or more metal cations M in an amount in the range of from 0.01 to 10 weight-% based on 100 weight-% of Si in the zeolitic material calculated as SiO₂, preferably in the range of from 0.05 to 7 weight-%, more preferably in the range of from 0.1 to 5 weight-%, more preferably in the range of from 0.5 to 4.5 weight-%, more preferably in the range of from 1 to 4 weight-%, more preferably in the range of from 1.5 to 3.5 weight-%, and more preferably in the range of from 2 to 3 weight-%.
9. The zeolitic material of any one of embodiments 1 to 8, wherein 95 or more weight-% of the zeolitic material consists of Si, Al, O, H, and the one or more metal cations M, calculated based on the total weight of the zeolitic material, preferably 95 to 100 weight-%, more preferably 97 to 100 weight-%, more preferably 99 to 100 weight-%.
10. The zeolitic material of any one of embodiments 1 to 8, wherein 95 or more weight-% of the framework of the zeolitic material consists of Si, Al, O, and H, based on the total weight of the framework of the zeolitic material, preferably 95 to 100 weight-%, more preferably 97 to 100 weight-%, and more preferably 99 to 100 weight-%.
11. The zeolitic material of any one of embodiments 1 to 10, wherein Y comprises, preferably consists of, Si, wherein the ²⁹Si MAS NMR of the zeolitic material comprises:
   a first peak in the range of from −99 to −102 ppm, preferably of from −99.5 to −101.5 ppm, more preferably of from −100 to −101.2 ppm, more preferably of from −100.3 to −100.9 ppm, and even more preferably of from −100.5 to −100.7 ppm;
   a second peak in the range of from −105.5 to −108 ppm, preferably of from −106 to −107.5 ppm, more preferably of from −106.3 to −107.3 ppm, more preferably of from −106.5 to −107.1 ppm, and even more preferably of from −106.7 to −106.9 ppm; and
   a third peak in the range of from −112.5 to −115 ppm, preferably of from −113 to −114.5 ppm, more preferably of from −113.3 to −114.3 ppm, more preferably of from −113.5 to −114.1 ppm, and even more preferably of from −113.7 to −113.9 ppm;
wherein preferably the ²⁹Si MAS NMR of the zeolitic material comprises only three peaks in the range of from −80 to −130 ppm.
12. The zeolitic material of any one of embodiments 1 to 11, wherein X comprises, preferably consists of, Al, wherein the ²⁷Al MAS NMR of the zeolitic material comprises:
   a peak in the range of from 55 to 58 ppm, preferably of from 55.5 to 57.5 ppm, more preferably of from 56 to 57 ppm, more preferably of from 56.6 to 56.8 ppm, and even more preferably of from 56.4 to 56.6 ppm,
wherein preferably the ²⁷Al MAS NMR of the zeolitic material comprises a single peak in the range of from −40 to 140 ppm.
13. The zeolitic material of any one of embodiments 1 to 12, wherein the BET surface area of the zeolitic material determined according to ISO 9277:2010 ranges from 100 to 850 m²/g, preferably from 300 to 800 m²/g, more preferably from 400 to 750 m²/g, more preferably from 500 to 700 m²/g, more preferably from 530 to 650 m²/g, more preferably from 550 to 620 m²/g, more preferably from 570 to 590 m²/g.
14. The zeolitic material of any one of embodiments 1 to 13, wherein the micropore volume of the zeolitic material determined according to ISO 15901-1:2016 ranges from 0.1 to 0.5 cm³/g, preferably from 0.15 to 0.4 cm³/g, more preferably from 0.2 to 0.35 cm³/g, more preferably from 0.23 to 0.32 cm³/g, more preferably from 0.25 to 0.3 cm³/g, and more preferably from 0.26 to 0.28 cm³/g.
15. The zeolitic material of any one of embodiments 1 to 14, wherein the zeolitic material is ITQ-24.
16. A process for the preparation of a zeolitic material having the IWR type framework structure, preferably of a zeolitic material according to any one of embodiments 1 to 15, wherein the process comprises
(1) preparing a mixture comprising one or more organotemplates as structure directing agents, one or more sources of YO₂, one or more sources of X₂O₃, and a solvent system;
(2) heating the mixture obtained in (1) for crystallizing a zeolitic material having the IWR type framework structure comprising YO₂ and X₂O₃ in its framework structure;
wherein the one or more organotemplates comprise an organodication of the formula (I):

R³R⁵R⁶N⁺—R¹-Q-R²—N⁺R⁴R⁷R⁸ (I);
wherein R¹ and R² independently from one another stand for (C₁-C₃)alkylene, preferably for C₁ or C₂ alkylene, more preferably for methylene or ethylene, and more preferably for methylene;
wherein Q stands for C₆-arylene, preferably for 1,4-C₆-arylene, and more preferably for benzene-1,4-diyl;
wherein R³ and R⁴ independently from one another stand for (C₁-C₄)alkyl, preferably (C₁-C₃)alkyl, more preferably for methyl or ethyl, and more preferably for methyl;
wherein R⁵, R⁶, R⁷, and R⁸ independently from one another stand for (C₁-C₆)alkyl, preferably (C₁-C₅)alkyl, more preferably (C₁-C₄)alkyl, more preferably (C₁-C₃)alkyl, more preferably for ethyl, isopropyl, or n-propyl, and more preferably for ethyl or n-propyl.
17. The process of embodiment 16, wherein the alkyl groups R⁵ and R⁶ are bound to one another to form one common alkylene chain, preferably a (C₅-C₇)alkylene chain, more preferably a (C₅-C₆)alkylene chain, more preferably a pentylene or hexylene chain, and more preferably a pentylene chain.
18. The process of embodiment 16 or 17, wherein the alkyl groups R⁷ and R⁸ are bound to one another to form one common alkylene chain, preferably a (C₅-C₇)alkylene chain, more preferably a (C₅-C₆)alkylene chain, more preferably a pentylene or hexylene chain, and more preferably a pentylene chain.
19. The process of any one of embodiments 16 to 18, wherein the organodication of the formula (I) has the formula (II):

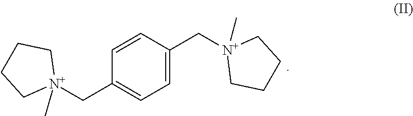 [SMILES: C[N+]1(Cc2ccc(C[N+]3(C)CCCC3)cc2)CCCC1]

20. The process of any one of embodiments 16 to 19, wherein the one or more organotemplates are provided as salts, preferably as one or more salts selected from the group consisting of halides, sulfate, nitrate, phosphate, acetate, hydroxide, and mixtures of two or more thereof, more preferably from the group consisting of bromide, chloride, hydroxide, sulfate, and mixtures of two or more thereof, wherein more preferably the one or more organotemplates are provided as hydroxides and/or bromides, and more preferably as hydroxides.
21. The process of any one of embodiments 16 to 20, wherein Y is selected from the group consisting of Si, Sn, Ti, Zr, Ge, and mixtures of two or more thereof, Y preferably being Si and/or Ti, wherein Y is more preferably Si.
22. The process of any one of embodiments 16 to 21, wherein X is selected from the group consisting of Al, B, In, Ga, and mixtures of two or more thereof, preferably from the group consisting of Al, B, Ga, and mixtures of two or more thereof, X more preferably being Al and/or B, wherein X is more preferably Al.
23. The process of any one of embodiments 16 to 22, wherein the mixture prepared in (1) further comprises seed crystals, wherein the seed crystals preferably comprise one or more all-silica zeolitic materials having the IWR type framework structure, wherein more preferably the seed crystals comprise all-silica ITQ-24, wherein more preferably one or more all-silica zeolitic materials having the IWR type framework structure is employed as the seed crystals, wherein more preferably all-silica ITQ-24 is employed as the seed crystals.

24. The process of any one of embodiments 16 to 23, wherein the mixture prepared in (1) further comprises seed crystals, wherein the seed crystals preferably comprise one or more zeolitic materials having the IWR type framework structure, and more preferably one or more zeolitic materials according to any one of embodiments 1 to 15 and 43, wherein more preferably one or more zeolitic materials having the IWR type framework structure is employed as the seed crystals, wherein more preferably one or more zeolitic materials according to any one of embodiments 1 to 15 and 43 is employed as the seed crystals.
25. The process of embodiment 24, wherein the amount of seed crystals comprised in the mixture prepared in (1) is in the range of from 0.1 to 15 mol % based on 100 mol % of the one or more sources of YO₂ calculated as YO₂, and preferably from 0.5 to 12 mol %, more preferably from 1 to 10 mol %, more preferably from 2 to 8 mol %, more preferably from 3 to 7 mol %, and more preferably from 5 to 6 mol %.
26. The process of any one of embodiments 16 to 25, wherein the mixture prepared in (1) and heated in (2) contains less than 5 weight-% of Ge calculated as GeO₂ and based on 100 weight-% of the one or more sources of YO₂ calculated as YO₂, preferably less than 3 weight-%, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.
27. The process of any one of embodiments 16 to 26, wherein the mixture prepared in (1) and heated in (2) contains less than 5 weight-% of B calculated as B₂O₃ and based on 100 weight-% of the one or more sources of X₂O₃ calculated as X₂O₃, preferably less than 3 weight-%, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.
28. The process of any one of embodiments 16 to 27, wherein the X₂O₃:YO₂ molar ratio of the one or more sources of X₂O₃ calculated as X₂O₃ to the one or more sources of YO₂ calculated as YO₂ in the mixture prepared in (1) and heated in (2) is in the range of from 5 to 1,500, preferably of from 10 to 1,200, more preferably of from 30 to 1,000, more preferably of from 50 to 900, more preferably of from 100 to 800, more preferably of from 200 to 700, more preferably of from 250 to 600, more preferably of from 300 to 500, and more preferably of from 350 to 400.
29. The process of any one of embodiments 16 to 28, wherein the organotemplate:YO₂ molar ratio of the one or more organotemplates to the one or more sources of YO₂ calculated as YO₂ in the mixture prepared in (1) and heated in (2) is in the range of from 0.01 to 1.5, preferably from 0.05 to 1.2, more preferably from 0.1 to 0.9, more preferably from 0.15 to 0.7, more preferably from 0.2 to 0.5, and more preferably from 0.25 to 0.3.
30. The process of any one of embodiments 16 to 29, wherein the mixture prepared in (1) further comprises one or more sources of fluoride, wherein preferably the fluoride:YO₂ molar ratio of the one or more sources of fluoride calculated as the element to the one or more sources of YO₂ calculated as YO₂ in the mixture prepared in (1) and heated in (2) is in the range of from 0.01 to 2, preferably from 0.05 to 1.5, more preferably from 0.1 to 1, more preferably from 0.3 to 0.8, and more preferably from 0.5 to 0.6.
31. The process of embodiment 30, wherein the one or more sources of fluoride is selected from fluoride salts, HF, and mixtures of two or more thereof, preferably from the group consisting of alkali metal fluoride salts, HF, and mixtures of two or more thereof, wherein more preferably the one or more sources of fluoride comprise HF, wherein more preferably HF is employed as the one or more sources of fluoride.
32. The process of any one of embodiments 16 to 31, wherein heating in (2) is conducted for a duration in the range of from 10 min to 10 d, preferably from 30 min to 9 d, more preferably from 1 h to 8 d, more preferably from 2 h to 7 d, and more preferably from 3 h to 6 d, more preferably from 6 h to 5.5 d, more preferably from 0.5 to 5 d, more preferably from 1 d to 4.5 d, more preferably from 2 d to 4 d, and more preferably from 2.5 to 3.5 d.
33. The process of any one of embodiments 16 to 32, wherein heating in (2) is conducted at a temperature in the range of from 80 to 220° C., preferably of from 110 to 200° C., more preferably of from 130 to 190° C., more preferably of from 140 to 180° C., more preferably of from 150 to 170° C., and more preferably of from 155 to 165° C.
34. The process of any one of embodiments 16 to 33, wherein heating in (2) is conducted under autogenous pressure, preferably under solvothermal conditions, more preferably under hydrothermal conditions, wherein preferably heating in (2) is performed in a pressure tight vessel, preferably in an autoclave.
35. The process of any one of embodiments 16 to 34, wherein the process further comprises
(3) isolating the zeolitic material obtained in (2),
and/or
(4) washing the zeolitic material obtained in (2) or (3),
and/or
(5) calcining the zeolitic material obtained in (2), (3), or (4),
and/or
(6) subjecting the zeolitic material obtained in (2), (3), (4), or (5) to an ion-exchange procedure with one or more metal cations M,
wherein the steps (3) and/or (4) and/or (5) and/or (6) can be conducted in any order, and
wherein one or more of said steps is preferably repeated one or more times.
36. The process of embodiment 35, wherein the one or more metal cations M are selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably from the group consisting of Sr, Cr, Mg, Ca, Mo, Fe, Co, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Cr, Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, and more preferably from the group consisting of Fe, Cu, Mg, Ca, Zn, Mo, La, Ce, Pr, Nd, Sm, Eu, Gd, Tb, Dy, Ho, Er, Tm, Yb, Lu, Y, Sc, and mixtures of two or more thereof, wherein the one or more metal cations M are located at the ion-exchange sites of the framework structure of the zeolitic material.
37. The process of embodiment 35 or 36, wherein calcination in (5) is conducted for a duration in the range of from 0.5 to 15 h, preferably of from 1 to 10 h, more preferably of from 2 to 8 h, more preferably of from 3 to 7 h, more preferably of from 3.5 to 6.5 h, more preferably of from 4 to 6 h, and more preferably of from 4.5 to 5.5 h.
38. The process of any one of embodiments 35 to 37, wherein calcination in (5) is conducted at a temperature in the range of from 300 to 800° C., preferably of from 350 to 700° C., more preferably of from 400 to 650° C., more preferably of from 450 to 600° C., and more preferably of from 500 to 550° C.
39. The process of any one of embodiments 16 to 38, wherein the one or more sources for YO₂ comprises one or more compounds selected from the group consisting of fumed silica, silica hydrosols, reactive amorphous solid silicas, silica gel, silicic acid, water glass, sodium metasilicate hydrate, sesquisilicate, disilicate, colloidal silica, silicic acid esters, and mixtures of two or more thereof,
preferably from the group consisting of silica hydrosols, silica gel, silicic acid, water glass, sodium metasilicate hydrate, sesquisilicate, disilicate, colloidal silica, tetra(C₁-C₄)alkylorthosilicate, and mixtures of two or more thereof,
more preferably from the group consisting of silica hydrosols, silicic acid, tetra(C₂-C₃)alkylorthosilicate, and mixtures of two or more thereof,
wherein more preferably the one or more sources for YO₂ comprises tetraethylorthosilicate, wherein more preferably tetraethylorthosilicate is used as the one or more sources for YO₂.
40. The process of any one of embodiments 16 to 39, wherein the one or more sources for X₂O₃ comprises one or more compounds selected from the group consisting of alumina, aluminates, aluminum salts, and mixtures of two or more thereof, preferably from the group consisting of alumina, aluminum salts, and mixtures of two or more thereof, more preferably from the group consisting of alumina, aluminum tri(C₁-O₅)alkoxide, AlO(OH), Al(OH)₃, aluminum halides, preferably aluminum fluoride and/or chloride and/or bromide, more preferably aluminum fluoride and/or chloride, and even more preferably aluminum chloride, aluminum sulfate, aluminum phosphate, aluminum fluorosilicate, and mixtures of two or more thereof, more preferably from the group consisting of aluminum tri(C₂-C₄)alkoxide, AlO(OH), Al(OH)₃, aluminum chloride, aluminum sulfate, aluminum phosphate, and mixtures of two or more thereof, more preferably from the group consisting of aluminum tri(C₂-C₃)alkoxide, AlO(OH), Al(OH)₃, aluminum chloride, aluminum sulfate, and mixtures of two or more thereof, more preferably from the group consisting of aluminum tripropoxides, AlO(OH), aluminum sulfate, and mixtures of two or more thereof, wherein more preferably the one or more sources for X₂O₃ comprises aluminum triisopropoxide, and wherein more preferably aluminum triisopropoxide is used as the one or more sources for X₂O₃.
41. The process of any one of embodiments 16 to 40, wherein the solvent system is selected from the group consisting of optionally branched (C₁-C₄)alcohols, distilled water, and mixtures thereof, preferably from the group consisting of optionally branched (C₁-C₃)alcohols, distilled water, and mixtures thereof, more preferably from the group consisting of methanol, ethanol, distilled water, and mixtures thereof, wherein more preferably the solvent system comprises distilled water, wherein more preferably the solvent system consists of distilled water.
42. The process of embodiment 41, wherein the H₂O:YO₂ molar ratio of H₂O to the one or more sources of YO₂ calculated as YO₂ in the mixture prepared in (1) and heated in (2) is in the range of from 0.5 to 15, preferably from 1 to 10, more preferably from 1.5 to 5, and more preferably from 2 to 3.
43. A zeolitic material obtainable and/or obtained from the process of any one of embodiments 16 to 42.
44. A method for the conversion of oxygenates to olefins comprising
(i) providing a catalyst according to any one of embodiments 1 to 15 and 43;
   (ii) providing a gas stream comprising one or more oxygenates and optionally one or more olefins and/or optionally one or more hydrocarbons;
(iii) contacting the catalyst provided in (i) with the gas stream provided in (ii) and converting one or more oxygenates to one or more olefins and optionally to one or more hydrocarbons;
(iv) optionally recycling one or more of the one or more olefins and/or of the one or more hydrocarbons contained in the gas stream obtained in (iii) to (ii).
45. The method of embodiment 44, wherein the catalyst is provided as a fixed bed or as a fluidized bed.
46. The method of embodiment 44 or 45, wherein the gas stream provided in (ii) comprises one or more oxygenates selected from the group consisting of aliphatic alcohols, ethers, carbonyl compounds and mixtures of two or more thereof, preferably from the group consisting of (C₁-C₆) alcohols, di(C₁-C₃)alkyl ethers, (C₁-C₆) aldehydes, (C₂-C₆) ketones and mixtures of two or more thereof, more preferably consisting of (C₁-C₄) alcohols, di(C₁-C₂)alkyl ethers, (C₁-C₄) aldehydes, (C₂-C₄) ketones and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone and mixtures of two or more thereof, more preferably from the group consisting of methanol, ethanol, dimethyl ether, diethyl ether, ethyl methyl ether and mixtures of two or more thereof, the gas stream more preferably comprising methanol and/or dimethyl ether, and more preferably dimethyl ether or a mixture of dimethyl ether and methanol.
47. The method of any one of embodiments 44 to 46, wherein the content of oxygenates in the gas stream provided in (ii) is in the range from 2 to 100% by volume based on the total volume, preferably from 3 to 99% by volume, more preferably from 4 to 95% by volume, more preferably from 5 to 80% by volume, more preferably from 6 to 50% by volume, more preferably from 7 to 40% by volume, more preferably from 8 to 30% by volume, more preferably from 9 to 20% by volume, and more preferably from 10 to 15% by volume.
48. The method of any one of embodiments 44 to 47, wherein the gas stream provided in (ii) comprises water, wherein the water content in the gas stream provided in (ii) is preferably in the range from 5 to 60% by volume, more preferably from 10 to 50% by volume, more preferably from 20 to 45% by volume, and more preferably from 30 to 40% by volume.
49. The method of any one of embodiments 44 to 48, wherein the gas stream provided in (ii) further comprises one or more diluting gases, preferably one or more diluting gases in an amount ranging from 0.1 to 90% by volume, more preferably from 1 to 85% by volume, more preferably from 5 to 80% by volume, more preferably from 10 to 75% by volume, more preferably from 20 to 70% by volume, more preferably from 40 to 65% by volume, more preferably from 50 to 60% by volume.
50. The method of any one of embodiments 44 to 49, wherein the one or more diluting gases are selected from the group consisting of H₂O, helium, neon, argon, krypton, nitrogen, carbon monoxide, carbon dioxide, and mixtures of two or more thereof, preferably from the group consisting of H₂O, argon, nitrogen, carbon dioxide, and mixtures of two or more thereof, wherein more preferably the one or more diluting gases comprise H₂O, wherein more preferably the one or more diluting gases is H₂O.
51. The method of any one of embodiments 44 to 50, wherein the contacting according to (iii) is effected at a temperature in the range from 200 to 700° C., preferably from 250 to 650° C., more preferably from 300 to 600° C., more preferably from 350 to 550° C., more preferably from 400 to 500° C., and more preferably from 425 to 475° C.
52. The method of any one of embodiments 44 to 51, wherein the contacting according to (iii) is effected at a pressure in the range from 0.1 to 50 bar, preferably from 0.3 to 30 bar, more preferably from 0.5 to 20 bar, more preferably from 1 to 15 bar, more preferably from 1.3 to 10 bar, more preferably from 1.5 to 7 bar, more preferably from 1.8 to 5 bar, more preferably from 2.0 to 3.0 bar, more preferably from 2.2 to 2.8 bar, more preferably from 2.4 to 2.6 bar.
53. The method of any one of embodiments 44 to 52, wherein the method is a continuous method, wherein the gas hourly space velocity (GHSV) in the contacting in (iii) is preferably in the range from 500 to 30,000 h⁻¹, preferably from 1,000 to 20,000 h⁻¹, more preferably from 1,500 to 10,000 h⁻¹, more preferably from 2,000 to 5,000 h⁻¹, more preferably from 2,200 to 3,000 h⁻¹ and more preferably from 2,400 to 2,600 h⁻¹.
54. The method of any one of embodiments 44 to 53, wherein the one or more olefins and/or one or more hydrocarbons optionally provided in (ii) and/or optionally recycled to (ii) comprise one or more selected from the group consisting of ethylene, (C₄-C₇)olefins, (C₄-C₇)hydrocarbons, and mixtures of two or more thereof, and preferably from the group consisting of ethylene, (C₄-C₅)olefins, (C₄-C₅)hydrocarbons, and mixtures of two or more thereof.
55. Use of a zeolitic material according to any one of embodiments 1 to 15 and 43 as a molecular sieve, as an adsorbent, for ion-exchange, or as a catalyst and/or as a catalyst support, preferably as a catalyst for the selective catalytic reduction (SCR) of nitrogen oxides NOₓ; for the oxidation of NH₃, in particular for the oxidation of NH₃ slip in diesel systems; for the decomposition of N₂O; as an additive in fluid catalytic cracking (FCC) processes; and/or as a catalyst in organic conversion reactions, preferably as a hydrocracking catalyst, as an alkylation catalyst, as an isomerization catalyst, or as a catalyst in the conversion of alcohols to olefins, and more preferably in the conversion of oxygenates to olefins.
56. The use of embodiment 55, wherein the zeolitic material is used in a methanol-to-olefin process (MTO process), in a dimethylether to olefin process (DTO process), methanol-to-gasoline process (MTG process), in a methanol-to-hydrocarbon process, in a methanol to aromatics process, in a biomass to olefins and/or biomass to aromatics process, in a methane to benzene process, for alkylation of aromatics, or in a fluid catalytic cracking process (FCC process), preferably in a methanol-to-olefin process (MTO process) and/or in a dimethylether to olefin process (DTO process), and more preferably in a methanol-to-propylene process (MTP process), in a methanol-to-propylene/butylene process (MT3/4 process), in a dimethylether-to-propylene process (DTP process), in a dimethylether-to-propylene/butylene process (DT3/4 process), and/or in a dimethylether-to-ethylene/propylene (DT2/3 process).

### DESCRIPTION OF THE FIGURES

FIG. 1 displays the ²⁹Si MAS NMR spectrum of the as-synthesized Al-IWR-200 zeolite obtained according to Example 1.

FIG. 2 displays the XRD patterns of the aluminosilicate IWR zeolite obtained from starting gels with SiO₂/Al₂O₃ ratios of (a) 30, (b) 150, and (c) 400, respectively.

FIG. 3 displays the ²⁷Al MAS NMR of the aluminosilicate IWR zeolite obtained from starting gels with SiO₂/Al₂O₃ ratios of (a) 30 (Example 2), (b) 150 (Example 3), and (c) 400 (Example 4), respectively.

FIG. 4 displays the SEM images of the aluminosilicate IWR zeolite obtained from starting gels with SiO₂/Al₂O₃ ratios of (a) 30 (Example 2), (b) 150 (Example 3), and (c) 400 (Example 4), respectively.

FIG. 5 displays the XRD patterns of the (a) Al-IWR-200, (b) H—Al-IWR-200, and (c) hydrothermally aged H—Al-IWR-200 zeolite as respectively obtained in Example 1.

FIG. 6 displays the XRD patterns of the (a) Ge—Al-IWR, (b) H—Ge—Al-IWR, and (c) hydrothermally aged H—Ge—Al-IWR zeolite as respectively obtained in Comparative Example 1.

FIG. 7 shows the dependencies of methanol conversion and product selectivities on the reaction time in MTO conducted in Example 10 over the H—Al-IWR-400 zeolite in the product at 480° C. (▪: conversion rate of methanol; ♦: C1; Δ: C2; ▴: C2═; ⋆: C3; ★: C3═; ○: C4; ● (black): C4═; ● (dark grey): C5+).

FIG. 8 shows the dependencies of methanol conversion and product selectivities on reaction time in MTO conducted in Example 10 over the aluminosilicate ZSM-5 zeolite at 480° C. (▪: conversion rate of methanol; ♦: C1; Δ: C2; ▴: C2═; ⋆: C3; ★: C3═; ○: C4; ● (lower values): C4═; ● (higher values): C5+).

### EXPERIMENTAL

### Characterization Via X-Ray Diffraction Analysis

X-ray powder diffraction (XRD) patterns were measured with a Rigaku Ultimate VI X-ray diffractometer (40 kV, 40 mA) using CuKα (λ=1.5406 Å) radiation.

### Characterization Via Solid State NMR

Solid MAS NMR was performed on a Bruker AVANCE-III 400 spectrometer. Magic angle spinning (MAS) experiments were performed on 3.2 mm MAS probes at a spinning speed of 15 kHz. The ²⁷Al signals were referenced to 1 M Al(NO₃)₃ solution at 0 ppm. The ²⁹Si signals were referenced to TMS at 0 ppm.

### Characterization Via SEM and TEM

Scanning electron microscopy (SEM) experiments were performed on Hitachi SU-1510 electron microscopes. Transmission electron microscopy (TEM) experiments were conducted on a JEOL JEM-2100P at 200 kV.

### Characterization of Surface Area and Porosity Characteristics

The N₂ sorption isotherms at the temperature of liquid nitrogen were measured using Micromeritics ASAP 2020M and Tristar system.

### Catalytic Testing in MTO

MTO reaction was performed in a fixed-bed reactor at an atmospheric pressure. The reaction temperature was at 480° C. The zeolite catalyst (0.50 g, 20-40 mesh) was pretreated in flowing nitrogen at 500° C. for 2 h and cooled down to reaction temperature. The methanol was injected into the catalyst bed by a pump with weight hourly space velocity (WHSV) of 1 h⁻¹. The product was analyzed by online gas chromatography (Agilent 6890N) with FID detector using PLOT-Al₂O₃ column.

### Materials For Synthesis

p-xylylene dibromide (C₈H₈Br₂, 97%, Aladdin Chemistry Co., Ltd.), tetraethylorthosilicate (C₈H₂₀O₄Si, TEOS, 99%, Aladdin Chemistry Co., Ltd.), hydrofluoric acid (HF, AR, 40%, Aladdin Chemistry Co., Ltd.), 1-methylpyrrolidine (C₅H₁₁N, 98%, Aladdin Chemistry Co., Ltd.), aluminum isopropoxide (C₉H₂₁O₃Al, CP, Sinopharm Chemical Reagent Co., Ltd.), acetonitrile (C₂H₃N, AR, 99%, Sinopharm Chemical Reagent Co., Ltd.), germanium oxide (GeO₂, 99.999%, Aladdin Chemistry Co., Ltd.), Beta zeolite (SiO₂/Al₂O₃=27.4, Tianjing Nankai Catalysts Co., Ltd.), diethyldimethylammonium hydroxide solution (DEDMAOH, 25wt % in water, Kente Catalysts Inc.), sodium metaaluminate (NaAlO₂, AR, 99%, Sinopharm Chemical Reagent Co., Ltd.), solid silica gel (SiO₂, 98%, Qingdao Haiyang Chemical Reagent Co., Ltd.), sodium hydroxide (NaOH, AR, 96%, Sinopharm Chemical Reagent Co., Ltd.), colloidal silica (40 wt % SiO₂ in water, Sigma-Aldrich Co., Ltd.), n-butylamine (C₄H₁₁N, Aladdin Chemistry Co., Ltd.).

### Reference Example 1: Synthesis of p-xylylene-bis((N-methyl)N-pyrrolidinium) hydroxide

In a typical example for the synthesis of the organotemplate, 13.2 g p-xylylene dibromide was dissolved in 250 mL acetonitrile, then 10.6 g 1-methylpyrrolidine was added, stirring for 48 h under reflux. After cooling to the room temperature, the mixture was filtrated and washed with acetonitrile three times. The solid was dried under vacuum condition overnight. The bromide cation was converted to hydroxide form using hydroxide exchange resin in water, and the obtained solution was titrated using 0.1 M HCl as titration.

### Comparative Example 1: Synthesis of an Germanosilicate Zeolite Having an IWR Type Framework Structure

In a typical example for the synthesis of Ge—Al-IWR zeolite, tetraethylorthosilicate (TEOS) was added into the solution of diethyldimethylammonium hydroxide solution (DEDMAOH, 25 wt % in water) in the 25 mL beaker, then germanium oxide and Beta zeolite (SiO₂/Al₂O₃=27.4, used as an aluminum source) were added one by one into above solution. After stirring overnight, the excess water and ethanol were evaporated, it was obtained a mixture with the molar ratio composition at 0.5 DEDMAOH:SiO₂:0.5 GeO₂:0.007 Al₂O₃:5.5 H₂O. The gel was transferred into Teflon line, sealed in a stainless steel autoclave, and then placed in a rotating oven and heated at 175° C. for 7 days. The final products were filtrated, washed with deionized water and dried overnight at 100° C. This sample was designated as Ge—Al-IWR. The organic template in the product was removed by calcining at 550° C. for 5 h in air. The calcined product was denoted as H—Ge—Al-IWR. After hydrothermal treatment of H—Ge—Al-IWR zeolite product at 800° C. with 10% H₂O for 4 h, the aged H—Ge—Al-IWR zeolite product was obtained.

### Comparative Example 2: Synthesis of ZSM-5 Zeolite Having an MFI Type Framework Structure

In a typical example for the synthesis of aluminosilicate ZSM-5 zeolite, 0.14 g of NaOH and 0.007 g of NaAlO₂ were dissolved in 4.5 g of deionized water. After stirring for 0.5 h, 0.365 g of n-butylamine was added into the above gel, followed by the addition of 1.0 g of solid silica gel. After stirring for another 2 h, the final gel was transferred into Teflon line, sealed in a stainless steel autoclave, and crystallized at 140° C. for 2 days. The solids were filtrated, washed with deionized water, dried overnight at 100° C. The sample was calcined at 550° C. for 5 h to remove organic template. The H-form of the product (H-ZSM-5) was prepared by ion-exchange with 1.0 M NH₄Cl solution three times and calcination at 450° C. for 4 h.

### Example 1: Direct Synthesis of an Aluminosilicate Zeolite Having an IWR Type Framework Structure

In a typical example for the synthesis of Al-IWR zeolite, tetraethylorthosilicate (TEOS) was added into a solution of p-xylylene-bis((N-methyl)N-pyrrolidinium) hydroxide in a 25 mL beaker, and then aluminum isopropoxide was added to this mixture. After stirring for 12 h, a clear solution was formed. After hydrofluoric acid was added to the above solution, the beaker was put into oven with the temperature of 80° C. for evaporating excess water and ethanol, the final molar compositions of the mixtures were 1.0 SiO₂:0.25 OSDA1:x Al₂O₃:0.5 HF:2 H₂O. At last, 6% of pure silica IWR seeds (mass ratios of seeds to the silica source) was added to the above mixtures and then the mixtures were ground. After grinding, the powder was transferred into Teflon line and sealed, crystallizing at 160° C. for 72 h under rotation condition (50 rpm). The final product was obtained by filtering, washing with deionized water, and subsequently drying overnight at 100° C. These samples were designated as Al-IWR-1/x. The organic template in the product was removed by calcining in air at 550° C. for 5 h. The calcined product was denoted as H—Al-IWR-1/x. After hydrothermal treatment of H—Al-IWR-200 zeolite at 800° C. with 10% H₂O for 4 h, the aged H—Al-IWR-1/x zeolite product was obtained.

As a typical example, the as-synthesized aluminosilicate IWR zeolite with the ratio of SiO₂/Al₂O₃ ratio at 200 in the starting gel is investigated. The X-ray diffraction pattern of the as-synthesized Al-IWR-200 zeolite shows a series of characteristic peaks associated with IWR structure, which are in good agreement with those of simulated XRD pattern of the IWR zeolite. N₂ sorption isotherms of the H—Al-IWR-200 zeolite product afford a BET surface area of 580 m²/g and a micropore volume of 0.27 cm³/g, which are higher than those of corresponding germanosilicate IWR zeolite These results should be related to the difference in thermal stability, where that aluminosilicate IWR zeolite is stable for calcination at 550° C., while germanosilicate IWR zeolite might be partially destroyed by the same calcination. Inductively coupled plasma (ICP) analysis of Si/Al of the Al-IWR zeolite affords a value of 85, corresponding to a silica to alumina molar ratio of 170.

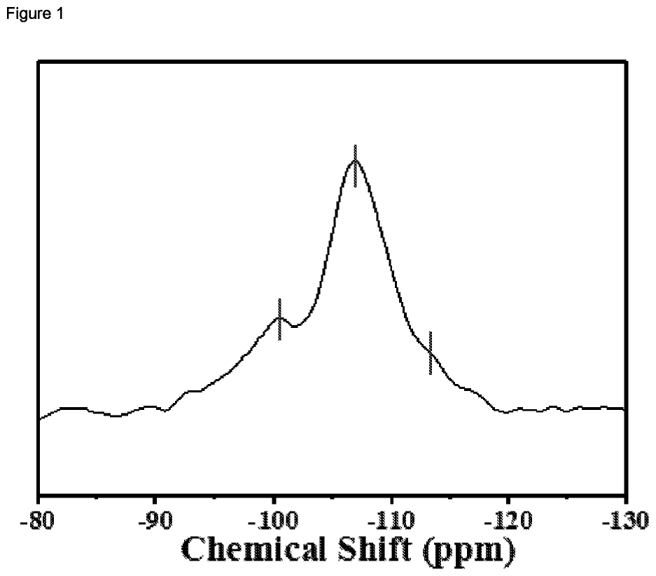

In FIG. 1, the ²⁹Si MAS NMR spectrum of the as-synthesized Al-IWR-200 zeolite is displayed, showing peaks with the chemical shift at −113.8, −106.8, and −100.6 ppm associated with Si(4Si), Si(4Si), and Si(3Si) respectively. The ²⁷Al MAS NMR spectrum of the as-synthesized aluminosilicate zeolite exhibits one signal with the chemical shift at 56.5 ppm associated with aluminum in the zeolite framework. This result demonstrates that all of the aluminum species have been successfully incorporated into the framework of IWR zeolite.

### Examples 2-4: Direct Synthesis of an Aluminosilicate Zeolites Having an IWR Type Framework Structure With Varying Silica to Alumina Ratios of the Synthesis Gel

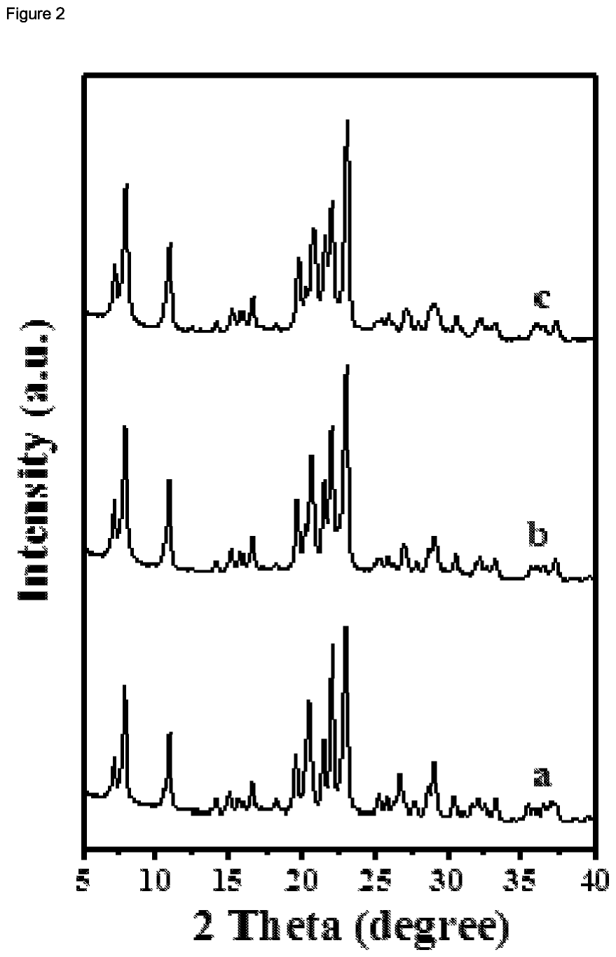

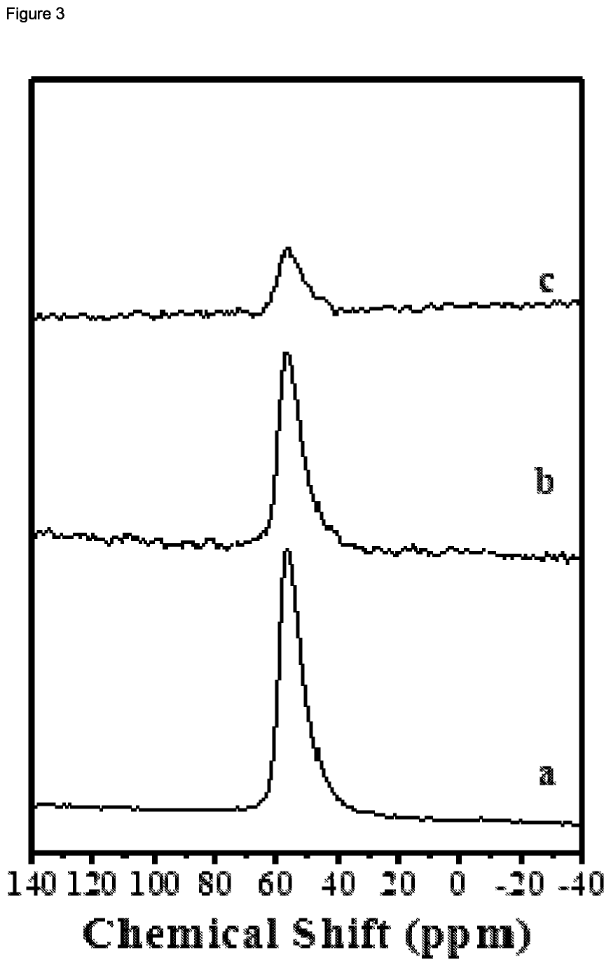

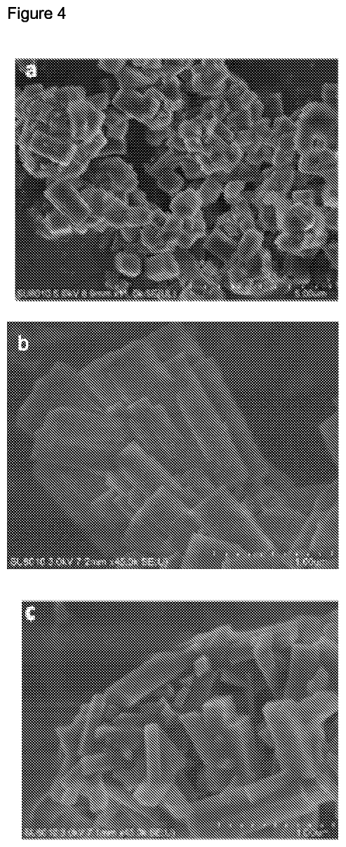

Example 1 was repeated, wherein the SiO₂/Al₂O₃ ratios of 30 (Example 2), 150 (Example 3), and 400 (Example 4) were respectively used in the starting gels for the synthesis of aluminosilicate IWR zeolite. FIGS. 2 to 4 exhibit XRD patterns (FIG. 2), ²⁷Al MAS NMR spectra (FIG. 3), and SEM images (FIG. 4) of aluminosilicate IWR zeolites with the aforementioned different SiO₂/Al₂O₃ ratios in the starting gels, showing that all of the products have very high crystallinity. More specifically, the aforementioned SEM images display that all of the products have perfect crystalline morphology; the aforementioned ²⁷Al MAS NMR spectra exhibit that all of the products have only a single peak with the chemical shift at 56.5 ppm associated with the signals of tetravalently-coordinated aluminum species. Results from ICP analysis displayed in Table 1 show that the SiO₂/Al₂O₃ ratios of the obtained products are close to those of the respective starting gels. Notably, considering the theoretical ratio of SiO₂/Al₂O₃ in this aluminosilicate zeolite of at least at 26, it is noted that the direct synthesis of aluminosilicate IWR zeolite according to the present invention almost realizes the minimum of SiO₂/Al₂O₃ (see Table 1: SAR=30 for Example 2).

### Examples 5-7: Direct Synthesis of Aluminosilicate Zeolites Having an IWR Type Framework Structure With Varying H₂O/SiO₂ Ratios of the Synthesis Gel

In the synthesis of aluminosilicate IWR zeolite, it is found that the addition of all silica IWR zeolite seeds and the ratio of H₂O/SiO₂ in the starting gel strongly influence the crystallization (see Table 1). Thus, Example 1 was repeated, wherein the H₂O/SiO₂ ratios of 10 (Example 5), 5 (Example 6), and 1 (Example 7) were respectively used in the starting gels for the synthesis of aluminosilicate IWR zeolite. Furthermore, when the ratio of H₂O/SiO₂ is 10.0, a zeolitic material of the MTW type framework structure is obtained as the main product (see Example 5 in Table 1); when the ratio of H₂O/SiO₂ is ranged from 1.0 to 5.0, the aluminosilicate IWR zeolites successfully synthesized (see Examples 6 and 7 in Table 1).

Example 8: Direct Synthesis of Aluminosilicate Zeolites Having an IWR Type Framework Structure Without Seeds in the Synthesis Gel

Example 1 was repeated, wherein no seeding material was added to the synthesis gel. In general when the IWR zeolite seeds are added, a product with high crystallinity is obtained. When the IWR zeolite seeds are not employed in the reaction mixture, a layered material is obtained in addition to the zeolitic material of the IWR type framework structure (see Example 8 in Table 1).

TABLE 1
Reaction mixture compositions and characterization of the
crystallization products for Examples 1 to 8

| | SiO₂/ | H₂O/ | | | ICP |
|---|---|---|---|---|---|
| Run^{a} | Al₂O₃ | SiO₂ | Seeds^{b} (%) | Products^{c} | (SiO₂/Al₂O₃) |
| Example 1 | 200 | 2 | 6 | IWR | 170 |
| Example 2 | 30 | 2 | 6 | IWR | 30 |
| Example 3 | 150 | 2 | 6 | IWR | 120 |
| Example 4 | 400 | 2 | 6 | IWR | 270 |
| Example 5 | 200 | 10 | 6 | MTW | |
| Example 6 | 200 | 5 | 6 | IWR | |
| Example 7 | 200 | 1 | 6 | IWR | |
| Example 8 | 200 | 2 | 0 | Layer + IWR | |

^{a}Crystallized at 160° C. for 72 h under rotation condition (50 rpm), organotemplate/SiO₂ = 0.25, and HF/SiO₂ = 0.5.
^{b}Mass ratios of seeds to the silica source.
^{c}The phase appearing first is dominant.

### Example 9: Aging and Hydrothermal Testing Experiments

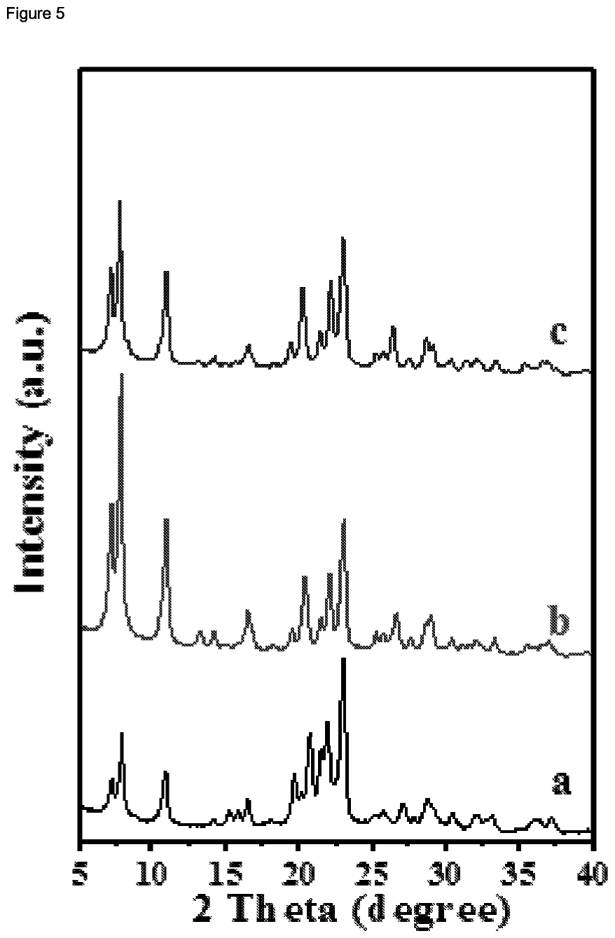

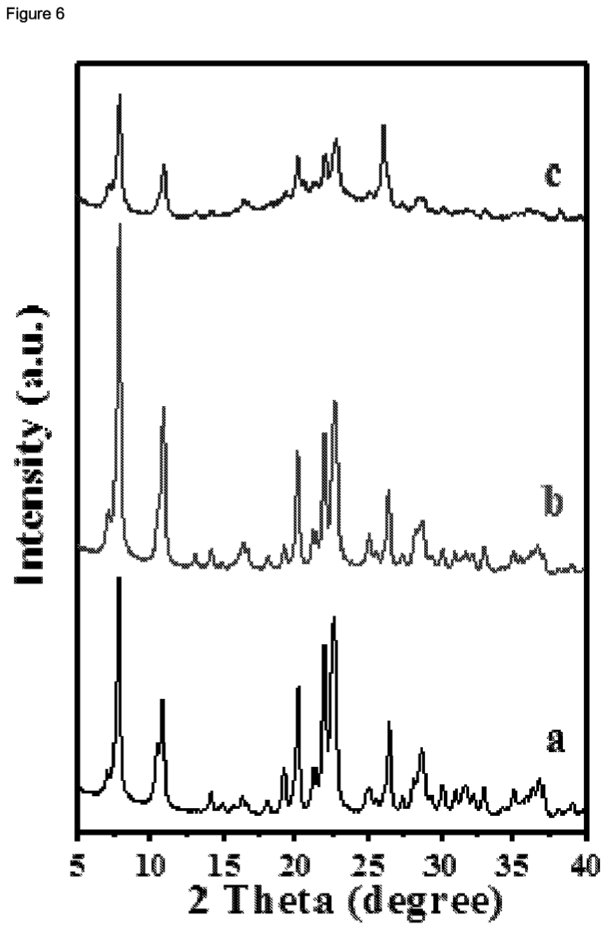

It is well known that the hydrothermal and thermal stabilities of zeolites are very important for catalytic applications. In general, the stability of aluminosilicate zeolite is much better than that of the germanosilicate zeolite containing aluminum, which was confirmed by the present experiments. Both of the as-synthesized Al-IWR-200 zeolite with the SiO₂/Al₂O₃ ratio of 170 according to Example 1 and the as-synthesized Ge—Al-IWR zeolite with the (GeO₂+SiO₂)/Al₂O₃ ratio of 196 prepared in Comparative Example 1 were calcined at 550° C. for 5 h. Very interestingly, although both show good crystallinity, the BET surface area and micropore volume are quite different. More specifically, the H—Ge—Al-IWR zeolite affords a BET surface area of 435 m²/g and a micropore volume of 0.17 cm³/g, which are lower than those of H—Al-IWR-200 zeolite (580 m²/g and 0.27 cm³/g). The lower BET surface area and micropore volume are mainly attributed to the micropore channel plugging by germanium removed from the zeolite framework at relatively high temperature. In addition, hydrothermal treatment of above two zeolites was performed at 800° C. with 10% H₂O for 4 h, leading to a significant decrease of the H—Ge—Al-IWR zeolite crystallinity (see FIG. 5). In contrast, the same treatment did not substantially change for the H—Al—IWR-200 zeolite crystallinity (see FIG. 6). Correspondingly, the BET surface area and micropore volume of H—Ge—Al-IWR zeolite (154 m²/g and 0.06 cm³/g) are much lower than those (511 m²/g and 0.21 cm³/g) of H—Al-IWR-200 zeolite. From the aforementioned results, it is concluded that the aluminosilicate IWR zeolite has much better hydrothermal and thermal stability than germanosilicate IWR zeolite. For an overview, the results from the measurement of the surface area and pore volume are shown in Table 2 below.

TABLE 2
Textural parameters of the IWR zeolites
before and after hydrothermal treatments.

| | | | Surface | | Pore volume | |
|---|---|---|---|---|---|---|
| | | | area (m²/g) | | (cm³/g) | |
| Run | Treatment | T/Al^{c} | S_{BET} | S_{Micro} | Vₜₒₜₐₗ | V_{Micro} |
| Ex. 1^{a} | Calcined | 85 | 580 | 574 | 0.33 | 0.27 |
| Ex. 1^{a} | Aging | | 511 | 446 | 0.32 | 0.21 |
| Comp. Ex. 1^{b} | Calcined | 98 | 435 | 379 | 0.32 | 0.17 |
| Comp. Ex. 1^{b} | Aging | | 154 | 136 | 0.19 | 0.06 |

^{a}H-Al-IWR-200 zeolite.
^{b}H-Ge-Al-IWR zeolite.
^{c}The molar ratios of T/Al (T = Si and Ge) were detected by ICP analysis.

### Example 10: MTO Testing

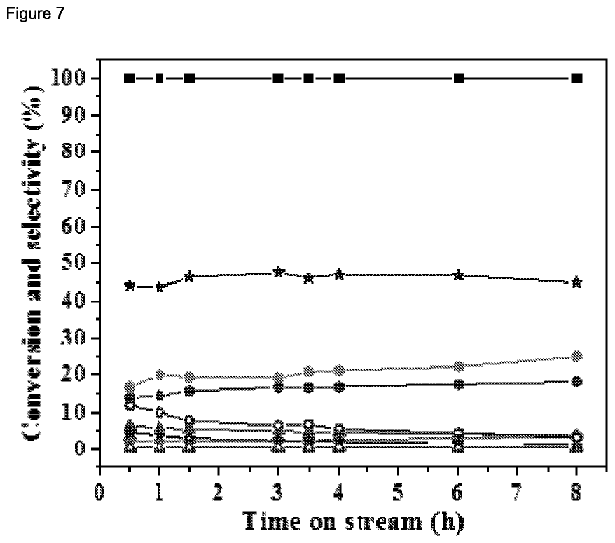

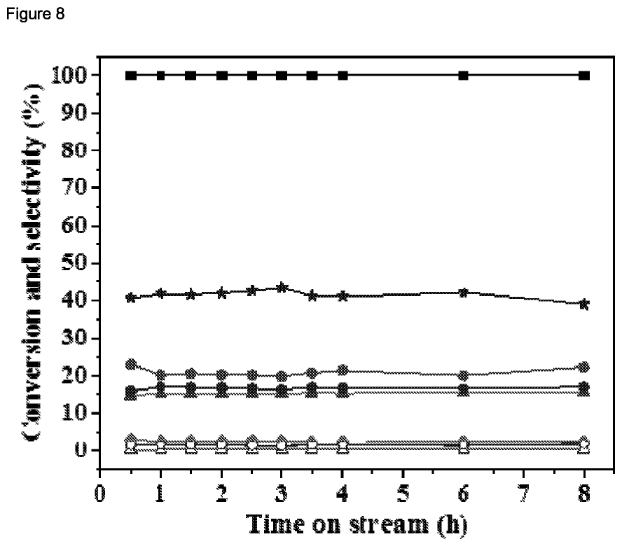

FIGS. 7 and 8 show catalytic conversions and product selectivities in the MTO reaction over the H—Al-IWR-400 zeolite from Example 4 and the H-ZSM-5 zeolite from Comparative Example 2 which have similar Si/Al ratios. Table 3 shows the results of reactions for 4 h. Clearly, the H—Al-IWR-400 zeolite exhibits a higher selectivity for propene and higher propene/ethene ratios than H-ZSM-5 zeolite, which is potentially important for the selective production of propylene in the industrial applications.

TABLE 3
Results from MTO testing at a reaction time of 4 hours at 480° C.

| | | Conv. | Selectivities (%) | | |
|---|---|---|---|---|---|
| Sample | SiO₂/Al₂O₃ | (%) | C₂̅ | C₃̅ | C₃̅/C₂̅ |
| H-AI-IWR- | 270 | 100 | 4.6 | 47.0 | 10.2 |
| 400 | | | | | |
| H-ZSM-5 | 242 | 100 | 15.3 | 41.1 | 2.7 |

Cited prior art:
EP 1 609 758 B1
Cantin, A. et al. in J. Am. Chem. Soc. 2006, 128, pp. 4216-4217
Shamzhy, M. et al. in Catal. Today 2015, 243, 76-84
CN 106698456 A
Simancas R. et al. in Science 2010, 330, pp. 1219-1222

1. A zeolitic material having the IWR type framework structure, wherein the zeolitic material comprises YO₂ and X₂O₃ in its framework structure, wherein Y is a tetravalent element and X is a trivalent element, and wherein the framework structure of the zeolitic material comprises less than 5 weight-% of Ge calculated as GeO₂ and based on 100 weight-% of YO₂ contained in the framework structure, and less than 5 weight-% of B calculated as B₂O₃ and based on 100 weight-% of X₂O₃ contained in the framework structure.

2. The zeolitic material of claim 1, wherein the zeolitic material comprises less than 3 weight-% of Ge calculated as GeO₂ and based on 100 weight-% of YO₂ contained in the framework structure.

3. The zeolitic material of claim 1, wherein the zeolitic material comprises less than 3 weight-% of B calculated as B₂O₃ and based on 100 weight-% of X₂O₃ contained in the framework structure.

4. The zeolitic material of claim 1, wherein Y is selected from the group consisting of Si, Sn, Ti, Zr, and mixtures of two or more thereof.

5. The zeolitic material of claim 1, wherein X is selected from the group consisting of Al, In, Ga, Fe, and mixtures of two or more thereof.

6. The zeolitic material of claim 1, wherein the YO₂:X₂O₃ molar ratio of the framework structure of the zeolitic material is in the range of from 5 to 1,000.

7. A process for the preparation of a zeolitic material having the IWR type framework structure, wherein the process comprises
(1) preparing a mixture comprising one or more organotemplates as structure directing agents, one or more sources of YO₂, one or more sources of X₂O₃, and a solvent system;
(2) heating the mixture obtained in (1) for crystallizing a zeolitic material having the IWR type framework structure comprising YO₂ and X₂O₃ in its framework structure;
wherein the one or more organotemplates comprise an organodication of the formula (I):
R³R⁵R⁶N⁺—R¹-Q-R²—N⁺R⁴R⁷R⁸ (I);
wherein R¹ and R² independently from one another stand for (C₁-C₃)alkylene;
wherein Q stands for C₆-arylene;
wherein R³ and R⁴ independently from one another stand for (C₁-C₄)alkyl;
wherein R⁵, R⁶, R⁷, and R⁸ independently from one another stand for (C₁-C₆)alkyl.

8. The process of claim 7, wherein the alkyl groups R⁵ and R⁶ are bound to one another to form one common alkylene chain.

9. The process of claim 7, wherein the alkyl groups R⁷ and R⁸ are bound to one another to form one common alkylene chain.

10. The process of claim 7, wherein the organodication of the formula (I) has the formula (II):

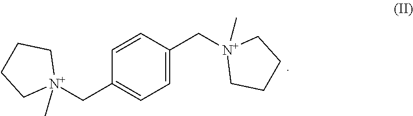 [SMILES: C[N+]1(Cc2ccc(C[N+]3(C)CCCC3)cc2)CCCC1]

11. The process of claim 7, wherein Y is selected from the group consisting of Si, Sn, Ti, Zr, Ge, and mixtures of two or more thereof.

12. The process of claim 7, wherein X is selected from the group consisting of Al, B, In, Ga, and mixtures of two or more thereof.

13. A zeolitic material obtainable and/or obtained from the process of claim 7.

14. A method for the conversion of oxygenates to olefins comprising
(i) providing a catalyst according to claim 1;
(ii) providing a gas stream comprising one or more oxygenates and optionally one or more olefins and/or optionally one or more hydrocarbons;
(iii) contacting the catalyst provided in (i) with the gas stream provided in (ii) and converting one or more oxygenates to one or more olefins and optionally to one or more hydrocarbons;
(iv) optionally recycling one or more of the one or more olefins and/or of the one or more hydrocarbons contained in the gas stream obtained in (iii) to (ii).

15. Use of a zeolitic material according to claim 13 as a molecular sieve, as an adsorbent, for ion-exchange, or as a catalyst and/or as a catalyst support.
